(19) United States
(12) Patent Application Publication
(10) Pub. No.: US 2024/0058165 A1
(43) Pub. Date: Feb. 22, 2024

(54) SYSTEM AND A METHOD FOR COOLING THE SCALP OF A USER

(71) Applicant: KEEPIT COLD CAP LTD., Kiryat Ono (IL)

(72) Inventors: Roy BENNATAN, Herzelia (IL); Avi SKOVLEVITZ, Herzelia (IL)

(21) Appl. No.: 18/355,535

(22) Filed: Jul. 20, 2023

(51) Int. Cl. A61F 7/10

(52) U.S. Cl. CPC A61F 7/10; A61F 2007/0008

(57) ABSTRACT

A system for cooling a head of a user to mitigate or prevent drug-induced hair-loss, including a hat, which includes an exterior layer, an interior layer, with an inflatable chamber therebetween, the inflatable chamber being functionally associated with an inflation pump. The hat includes a cooling assembly, reversibly attachable to the interior layer and including a housing defining pockets having cooling pads therein. Each cooling pad comprising a phase-changing-material adapted to obtain a temperature lower than −20° C. and to retain that temperature. A sensor is adapted to sense an environmental condition within the hat. A controller is adapted to receive input from the sensor, and to provide a notification when the sensed environmental condition is outside of a specific range. When the hat is donned by the user, the inflatable chamber is inflated sufficiently to ensure that the cooling assembly engages the scalp of the user.

### FIELD OF THE INVENTION

The present invention relates to systems and methods for cooling a scalp of a user, and specifically to systems and methods which cool the scalp of the user sufficiently to reduce or prevent drug-induced hair-loss during medical treatments such as chemotherapy.

### BACKGROUND OF THE INVENTION

Various medical treatments, such as chemotherapy and other treatments which include cytotoxic drugs, induce hair-loss. It is medically known that cooling of a region, such as a user's scalp during chemotherapy reduces or prevents hair-loss. For example, cooling of the scalp constricts the blood supply to hair follicles in the scalp, thereby diminishing or preventing the hair follicles receiving high cytotoxic drug concentrations during the initial phases of chemotherapy. Furthermore, the coldness itself reduces the metabolism of cells of the hair follicles and thus reduces the ability of the cytotoxic drugs to act on the hair follicles. The combined effect of constricting the blood supply to the hair follicles and reducing the metabolism of the cells of the hair follicles prevents or reduces the hair loss.

The prior art includes various types of head-cooling devices for cooling the head during chemotherapy treatments.

For example, some devices include gel- or ice-based cooling pads, which are placed within a helmet or other head wrapper, and placed on the user's head. However, such devices require frequent changing of the cooling pads (e.g. every half hour to 1 hour), and placing the now-heated pads in dry ice to re-cool. Consequently, the process is highly cumbersome. Additionally, devices using cooled gel packs or ice packs often do not reach, or do not sufficiently maintain, the desired degree of cooling of the user's scalp in order to reduce or prevent hair-loss.

As another example, some devices include a hat or helmet including channels for flow of a cooled fluid therethrough. The hat or helmet is fluidly connected to a cooling device, a fluid reservoir, and a pump, and the fluid flowing through the channels is constantly or periodically replaced and re-cooled, in order to maintain the desired temperature. This type of devices, while maintaining an appropriate cooling temperature, are typically large and cumbersome, and are prone to problems and malfunctions because of the many different parts that must interact with each other throughout the process.

This and more, for best treatment rests, the user's head must be cooled at the time of initiating the treatment and must continue to be cooled for several hours following completion of the treatment. Typically, the cooling process should begin approximately two hours prior to initiation of the chemotherapeutic treatment, and should terminate approximately two hours following completion of the treatment. As such, the user typically has to travel to the hospital or clinic where the treatment will be provided, and back home following the treatment, while their head is being cooled. The prior art systems requiring fluid flow through the hat or helmet are prohibitive in this regard, because they require an electrical connection for operation of the pump and cooling mechanism during the entire time the user's head is being cooled, and thus cannot conveniently be used in a vehicle during travel time. Similarly, devices which require frequent changing of cooling pads are inconvenient for use during travel, due to the need to repeatedly stop in order to replace the cooling pads.

There is thus a need in the art for a system and method for cooling a user's head before, during, and after chemotherapeutic treatments, in order to reduce or prevent hair-loss, which system and method are suitable for use in a variety of situations, such as during travel.

### SUMMARY OF THE INVENTION

The present invention relates to systems and methods for cooling a scalp of a user in order to reduce or prevent drug-induced hair-loss during medical treatments such as chemotherapy.

In accordance with an embodiment of the present invention, there is provided a system for cooling a head of a user during medical treatments so as to mitigate or prevent drug-induced hair-loss. The system includes a hat, which includes an exterior layer, an interior layer, and an inflatable chamber disposed between the exterior layer and the interior layer. The inflatable chamber is functionally associated with an inflation interface adapted for connection to an inflation pump. The hat further includes a cooling assembly, reversibly attachable to an interior surface of the interior layer. The cooling assembly includes a housing defining a plurality of pockets and a plurality of cooling pads, each comprising a phase-changing-material (PCM) adapted to obtain a predetermined temperature lower than −20° C. and to retain the predetermined temperature for a predetermined duration. The hat further includes at least one sensor adapted to sense an environmental condition within the hat. The hat additionally includes a first communication interface and a first controller, adapted to receive input from the at least one sensor, and to provide a notification to another device, via the first communication interface, when the sensed environmental condition is outside of a specific range. When the hat is donned by the user, the inflatable chamber is inflated sufficiently to ensure that the cooling assembly engages the scalp of the user.

In some embodiments, each cooling pad is adapted to obtain a predetermined temperature lower than −28° C.

In some embodiments, each cooling pad is adapted to retain the predetermined temperature of a duration of at least three hours.

In some embodiments, the at least one sensor comprises at least one temperature sensor adapted to provide to the first controller input regarding a temperature within the hat.

In some embodiments, the at least one sensor comprises at least one pressure sensor adapted to provide to the first controller input regarding a pressure applied by the hat to the scalp of the user.

In some embodiments, the hat further comprises a sizing assembly adapted to adjust at least one dimension of the hat so as to maintain the hat fixed on the user's head.

In some embodiments, the cooling assembly is devoid of fluid communication with other elements of the hat or outside the hat.

In some embodiments, the cooling assembly is devoid of electronic components.

In some embodiments, the system further includes a pumping unit, including a pump adapted to be in fluid communication with the inflation interface of the hat for inflation of the inflatable chamber, and a second communication interface adapted to be in communication with the first communication interface. The pumping unit may further include a user interface, adapted to receive input from a user and to provide output to the user, and a second controller, functionally associated with the pump, the second communication interface, and the user interface, for controlling operation thereof.

In some embodiments, upon the second controller receiving a signal from the first controller indicating that a temperature within the hat is above a predetermined temperature threshold, the second controller is adapted to provide to the user a notification, via the user interface, to replace the cooling pads in the cooling assembly.

In some embodiments, upon the second controller receiving a signal from the first controller indicating that a pressure temperature within the hat is below a predetermined pressure threshold, the second controller is adapted to automatically activate the pump to inflate the inflatable chamber of the hat.

In accordance with another embodiment of the present invention, there is provided a device for control of a hat for cooling a head of a user, the hat having: (i) an inflatable chamber; (ii) a cooling assembly; (iii) a pressure sensor; (iv) a temperature sensor; and (v) a communication interface. The device includes a pump adapted to be in fluid communication with the inflatable chamber of the hat, a user interface adapted to provide output to the user, and a controller. The controller is configured to receive temperature input from the temperature sensor, and upon identification of the temperature input being indicative of a temperature in the hat being above a predetermined temperature threshold, provide a notification to the user, via the user interface, to replace cooling units of the cooling assembly within the hat.

In some embodiments, the controller is further configured to receive pressure input from the pressure sensor in the hat, and upon identification of the pressure sensor being indicative of the pressure in the hat being below a predetermined pressure threshold, automatically activate the pump to re-inflate the inflatable chamber of the hat, thereby increasing the pressure therein.

In accordance with an embodiment of the present invention, there is provided a method of cooling the scalp of a user during a medical treatment so as to reduce or prevent drug-induced hair-loss, using the system described herein. The method includes at a first predetermined duration prior to initiation of the medical treatment, with the cooling assembly having cooling pads therein and being connected to the interior layer, donning the hat on the head of the user, and inflating the inflatable chamber. The method further includes retaining the hat on the head until receipt of a notification indicating that a temperature of the cooling assembly is above a temperature threshold. The method further includes, following receipt of the notification, replacing the cooling pads within the cooling assembly and re-donning the hat on the head of the user, and repeating the retaining, replacing, and re-donning steps until the passage of a second predetermined duration following completion of the medical treatment.

In some embodiments, the first predetermined duration is at least one hour.

In some embodiments, the second predetermined duration is at least four hours.

In some embodiments, the temperature threshold is −20° C.

In some embodiments, the temperature threshold is −25° C.

In some embodiments, the temperature threshold is −28° C.

### BRIEF DESCRIPTION OF THE FIGURES

The foregoing discussion will be understood more readily from the following detailed description of the invention, when taken in conjunction with the accompanying FIGS. 1-5), in which:

FIG. 1 is a block diagram of a system for cooling the head or scalp of a user according to embodiments of the disclosed technology;

FIGS. 2A and 2B are, respectively, a perspective view illustration and a sectional illustration of a hat forming part of the system of FIG. 1, according to an embodiment of the disclosed technology;

FIG. 3 is a perspective view illustration of a pumping unit forming part of the system of FIG. 1, according to an embodiment of the disclosed technology;

FIG. 4 is a flow chart of a method of user operation of the system of FIG. 1, according to an embodiment of the disclosed technology; and

FIG. 5 is a flow chart of a method of operation of control components of the system of FIG. 1, according to an embodiment of the disclosed technology.

### DESCRIPTION OF THE PREFERRED EMBODIMENTS

The principles of the inventive systems and methods for cooling a scalp of a user may be better understood with reference to the drawings and the accompanying description.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

For the purposes of the present disclosure, the term “substantially” is defined as “within 10% deviation of”.

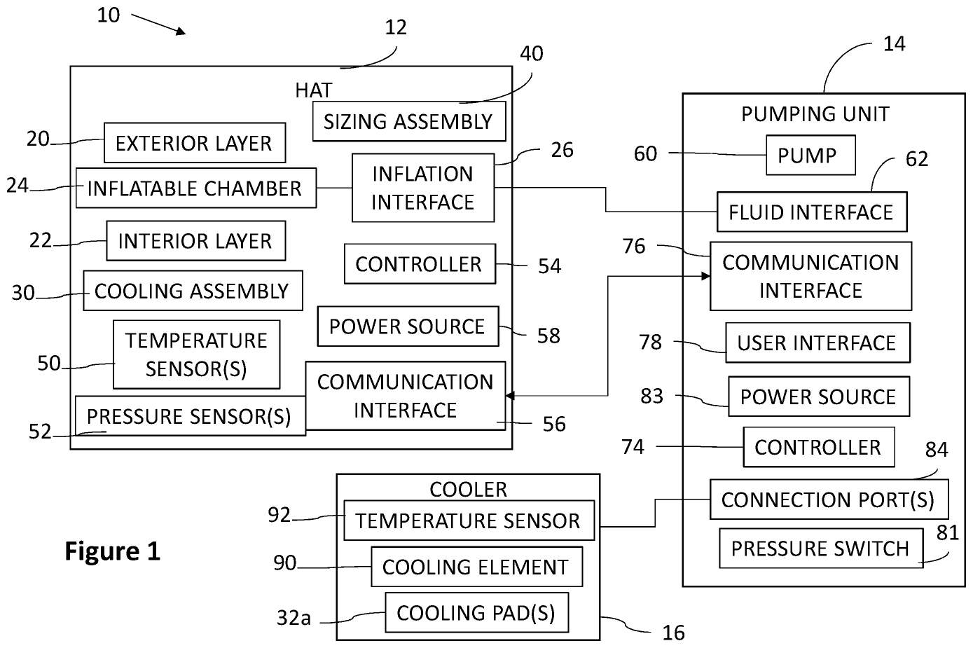

Reference is now made to FIG. 1, which is a block diagram of a system 10 for cooling the head, or scalp, of a user according to embodiments of the disclosed technology.

System 10 includes a hat or helmet 12, a pumping unit 14, and a cooler 16.

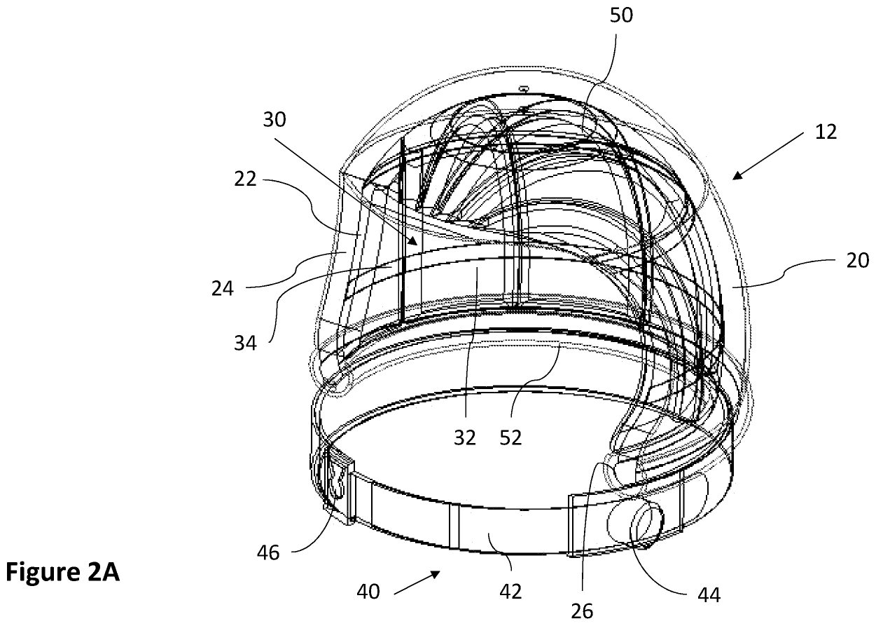

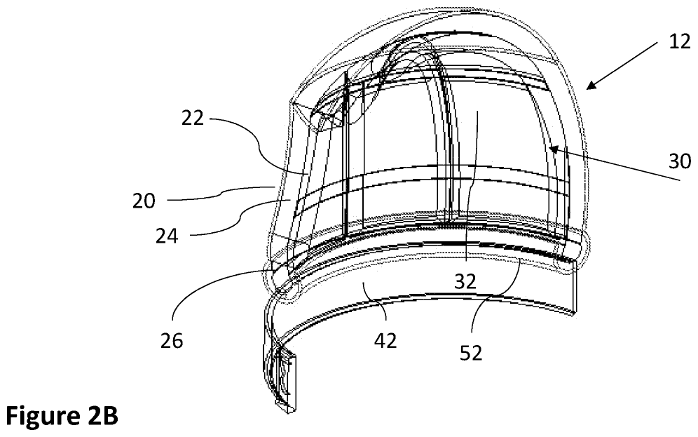

Hat 12 is described herein also with reference to FIGS. 2A and 2B, which are, respectively, a perspective view illustration and a sectional illustration of the hat.

Hat 12 includes an exterior layer 20, which may formed of an elastic material or a non-woven material. Typically, exterior layer 20 has some degree of elasticity. Hat 12 further includes an interior layer 22, such that an inflatable chamber 24 is formed between interior layer 22 and exterior layer 20. Inflatable chamber 24 is functionally associated with an inflation interface 26, adapted to be in fluid communication with pumping unit 14 for inflation of the inflatable chamber and/or deflation thereof. In some embodiments, interior layer 22 may include or may be formed of silicone.

As explained in further detail hereinbelow, inflatable chamber 24 may be inflated with a fluid, such as air, to increase the pressure within hat 12 and to ensure proper engagement between cooling elements of the hat and the scalp of the user. Typically, a single inflatable chamber 24 is used, to ensure even distribution of the pressure through the entire area of the hat.

Hat 12 further includes a cooling assembly 30, adapted to attach to an interior surface of interior layer 24, such that when hat 12 is worn on the head of a user, cooling assembly 30 engages the scalp. Cooling assembly 30 is formed of a plurality of pockets or chambers 31, each of which receives a cooling pad 32. Cooling assembly 30 is typically formed of silicone, and is adapted to assist in retaining the coldness of cooling pads 32 for an extended duration.

Cooling pads 32 typically contain, or are formed of, a phase changing material (PCM) configured to reach and retain a temperature in the range of −40° C. to −20° C., or −35° C. to −28° C. for a duration of at least two hours, at least three hours, or at least four hours. Typically, the PCM of cooling pads 32 is cooled using cooler 16, as explained in further detail hereinbelow.

In some embodiments, cooling assembly 30 and interior layer 24 include corresponding attachment interfaces 34 for rapid and convenient attachment of the cooling assembly to the interior surface of interior layer 24. For example, the attachment interfaces may include Velcro® hooks and loops materials, hooks and eyes, zippers, buttons, snaps, and the like.

It is a particular feature of the present invention that cooling assembly 30 is a stand-alone assembly, which is mechanically detachable from hat 12. While connected to hat 12, and in some embodiments at all times, cooling assembly 30 is devoid of fluid communication with pumping unit 14 or with any other pumping or cooling unit. In some embodiments, cooling assembly 30 is devoid of any form of fluid communication, and is fluidly isolated from all other components of system 10. Cooling assembly 30 is further devoid of any electrical or electronic components, and is devoid of connection or communication to any electrical or electronic components.

Exterior layer 22 of hat 12 is functionally associated with a sizing assembly 40, adapted to enable adjustment of the size of hat 12 to the head of a specific user. For example, sizing assembly 40 may include a rigid, or semi-rigid, cuff 42, adapted to wrap around the user's neck and to be adjusted to the size of the user's neck, so as to ensure that hat 12 does not move relative to the user's head during cooling of the head. A tightening button 44 allows for mild adjustments of the tightness of cuff 42, and a release button 46 enables immediate release of cuff 42 to facilitate donning and doffing of hat 12, as well as rapid removal of the hat in case of stress or discomfort.

Hat 12 further has mounted therein, or thereon one or more temperature sensors 50 adapted to sense a temperature within the hat, and may include one or more pressure sensors 52 adapted to sense pressure applied to the scalp of the user by the hat, or the pressure within inflatable chamber 24. Sensors 50 and 52 are functionally associated with a controller 54, which in turn is functionally associated with a communication interface 56 both being mounted in or on hat 12. Controller 54 is adapted to receive inputs from sensors 50 and 52, and to provide instructions or communication messages to pumping unit 14 or to a remote computing unit, as explained in further detail hereinbelow. A power source 58, which typically includes a battery such as a rechargeable battery, is disposed within hat 12 and powers electronic components thereof, such as sensors 50 and 52, controller 54, and communication interface 56.

The term “controller” as used herein means a computing device configured for monitoring, controlling, regulating and/or actuating one or more components, assemblies, stations, systems, or subsystems. A controller should be understood to include any or all of (and not exhaustively): one or more processors, one or more computer-readable media, e.g., transient and/or non-transient storage media, communications arrangements, a power source and/or a connection to a power source, and firmware and/or software. Controllers can be programmed in advance, e.g., by having program instructions stored in the computer-readable media for execution by one of more processors of the controller. Thus, a controller ‘configured’ to perform a function is equivalent herein to the controller being programmed, i.e., having access to stored program instructions for execution, to perform the function.

In some embodiments, hat 12 may have a pre-formed shape, such as a helmet. The shape and sizing of the hat may be adjustable using sizing assembly 40 and a degree of inflation of inflatable chamber 24.

In some embodiments, hat 12 may have a flat configuration, in which the exterior layer of the hat forms a plane or flat surface, and a donned configuration as shown in FIG. 2A. In such embodiments, hat 12 may further includes reversible connectors for transitioning of the hat between the two configurations.

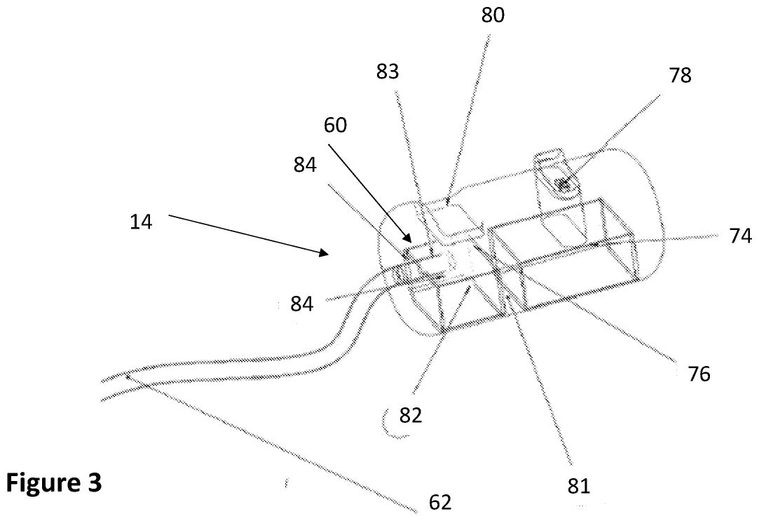

Pumping unit 14 is described herein also with reference to FIG. 3, which is a perspective view illustration of the pumping unit according to an embodiment of the disclosed technology

Pumping unit 14 includes a pump 60, functionally associated with a fluid interface 62. Fluid interface 62 is adapted to be in fluid communication with inflation interface 26 of hat 12, so that fluid (e.g. air) pumped by pump 60 can be used to inflate inflatable chamber 24. Fluid interface 62 and inflation interface 26 are reversibly connectable to each other, for example using a suitable pipe or tube 64 shown clearly in FIG. 3.

Pumping unit 14 further includes a controller 74, functionally associated with pump 60, with a communication interface 76 and with a user interface 78.

User interface 78 is adapted to receive user instructions for operation of system 10, such as instructions to inflate inflatable chamber 24. User interface 78 is further adapted to provide output to the user, for example via a display 80 forming part of the user interface or via an audio speaker 82. User interface 78 typically further includes an activation switch for activation and deactivation of operation of hat 12 and/or of system 10.

Communication interface 76 is adapted to communicate with communication interface 56 of hat 12 for communication between the hat and the pumping unit. Communication interface 76 may further be adapted to communicate with other remote device, such as a computing device or smart phone of the user (not explicitly shown) running a dedicated software application. Communication interfaces 56 and 76 may communicate with each other using any suitable protocol known in the art, such as via Bluetooth communication, Radio Frequency communication, Wi-Fi communication, and the like.

In some embodiments, pumping unit 14 further includes a pressure sensor or a pressure switch 81, adapted to assess the fluid pressure, or air pressure, in the fluid flow system of the pump 60 and inflatable chamber 24. Pressure switch 81 is functionally associated with controller 74, such that, when pressure switch 81 reports to the controller that there is a drop in pressure in the fluid flow system, controller 74 activates pump 60 to increase the pressure back to a desired pressure.

A battery, or another power source, 83, powers all the electronic components of pumping unit 14.

In some embodiments, pumping unit 14 may further include one or more connection ports 84 for connection to external devices. For example, connection port(s) 84 may include a USB connection port for direct connection to controller 54 of hat 12, or to a connection interface thereof. As another example, connection port(s) 84 may include a connection port for connection to cooler 16, for example for extraction of data relating to the interior of the cooler, as explained in further detail hereinbelow.

Cooler 16 is adapted to have disposed in a hollow thereof additional cooling pads 32a, typically identical to cooling pads 32 and adapted to fit into cooling assembly 30. The additional cooling pads 32a are stored in cooler 16 together with, or within, a cooling element 90, and are brought to and maintained at the desired temperature, ready for a time at which the cooling pads within cooling assembly 30 need to be switched.

In some embodiments, cooling element 90 may comprise a suitable chemical cooler, such as dry ice (solid carbon dioxide), or liquid nitrogen. Typically, cooling element 90 is devoid of electronic components, and is not electrically maintained at its cold temperature. Typically, cooling element 90 is maintained at, or is configured to maintain within cooler 16, a temperature in the range of −50° C. to −30° C.

Cooler 16 may further include a temperature sensor 92, adapted to sense the temperature within the cooler. The temperature sensor may be associated with a communication interface (not explicitly shown) for communication of temperature readings to communication interface 76 of pumping unit 14. Alternately, the temperature sensor may be associated with a connection interface adapted for direct connection to connection port(s) 84 for extraction of data from the temperature sensor by controller 74 of the pumping unit.

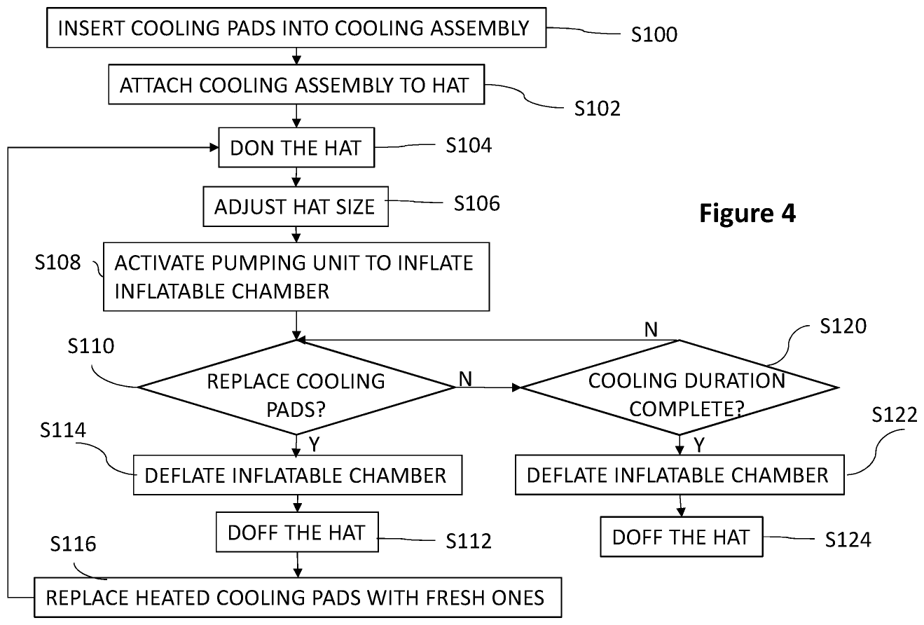

Reference is now made to FIG. 4, which is a flow chart of a method of user operation of system 10, according to an embodiment of the disclosed technology, and using the reference numerals used hereinabove with respect to FIGS. 1-3.

At step S100, the user removes cooled cooling pads 32 from cooler 16, and inserts them into the suitable pockets in cooling assembly 30. The user then attaches cooling assembly 30 to interior surface 22 of hat 12 at step S102, and dons the hat at step S104. In some embodiments, following donning of the hat, the user may adjust the hat size using sizing assembly 40, at step S106. In accordance with some embodiments of the disclosed technology, donning of the hat and initiating the cooling process takes place a predetermined duration prior to initiation of therapeutic treatment, such as half an hour to 2 hours prior to the beginning of the therapeutic treatment.

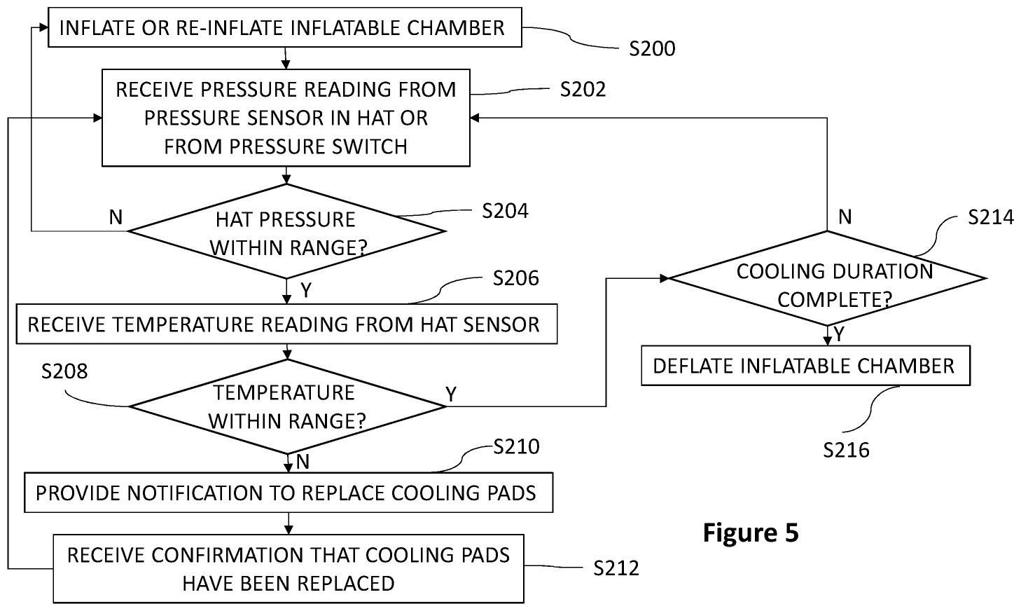

At step S108, the user activates pumping unit to pump fluid to inflate inflatable chamber 24. Control of the inflation process is automatic, as explained hereinbelow with respect to FIG. 5, so that it is sufficient for the user to give a single inflate command for the system to work as required.

The user then keeps the hat on their head, and periodically checks, at step S110 whether a notification is received via user interface 78 of pumping unit 14 that the cooling pads must be replaced. This may occur, for example, when the system identifies that the temperature within hat 12 is above a desired temperature, as explained hereinbelow with respect to FIG. 5.

If such a notification is received, at step S112 the user doffs hat 12, and, in some embodiments, also deflates the inflatable chamber at step S114. The user then replaces the heated cooling pads 32 with fresh cooling pads taken from cooler 16, at step S116. For example, replacement of the cooling pads may include detaching cooling assembly 30 from interior surface 22 of the hat, removing the heated cooling pads, from the cooling assembly and placing them in the cooler to cool, inserting cold cooling pads from the cooler into the cooling assembly, and re-attaching the cooling assembly to the hat.

The flow then returns to step S104, where the user once again dons the hat with the refreshed cooling pads. In some embodiments, in which the size of the hat was not changed when it was doffed by the user, there is no need to re-adjust the size of the hat at step S106. Similarly, in embodiments in which the hat was not deflated when it was doffed by the user, there is no need to re-inflate the inflatable chamber at step S108.

Alternately, if no notification to replace the pads is received, the user may replace the pads following the passage of a predetermined duration, such as 3 hours. Additionally, the user occasionally checks whether the cooling duration is complete, at step S120. For example, the cooling duration may be complete approximately 4 to 5 hours after completion of the chemotherapeutic treatment.

If the cooling treatment is complete, the user deflates the hat at step S122, and doffs it at step S124. Otherwise, the flow returns to step S110.

Reference is now made to FIG. 5, which is a flow chart of a method of operation of control components of system 10, and specifically of pumping unit 14, according to an embodiment of the disclosed technology.

At step S200, which occurs following activation of pumping unit 14, controller 74 drives pump 60 to inflate inflatable chamber 24 of the hat. At some later time, at step S202, controller 74 receives a pressure reading from pressure sensor(s) 52 within hat 12, or from pressure switch 81, which reading is indicative of a pressure within the hat or within the pumping system. Such a reading may be received periodically (e.g. every 1 second, every 15 seconds, every 30 seconds, every minute, every 2 minutes, every 5 minutes, etc) or intermittently. For example, the pressure reading may be received from pressure sensor(s) 52 via communication interfaces 56 and 76.

Controller 74 then evaluates whether the hat pressure is within range, at step S204. If the pressure within the hat is outside of the desired range, the flow returns to step S200, for re-inflation of the hat.

Otherwise, if the pressure in the system is within the desired range, the flow continues to step S206, where, at some time, controller 74 receives a temperature reading from temperature sensor(s) 50 in hat 12. Such a reading may be received periodically (e.g. every 1 second, every 15 seconds, every 30 seconds, every minute, every 2 minutes, every 5 minutes, etc) or intermittently. For example, the temperature reading may be received from temperature sensor(s) 50 via communication interfaces 56 and 76.

Controller 74 then evaluates whether the temperature is within range, at step S208. For example, the temperature is considered within range if it is the range of −40° C. to −20° C., or −35° C. to −28° C. If the temperature is out of range, and specifically higher than the desired range, at step S210 controller 74 notifies the user that the cooling pads must be replaced. In some embodiments, controller 74 may optionally receive a signal from the user following replacement of the cooling pads, at step S212. For example, such a signal may be provided via a suitable switch or button in user interface 78.

If at step S208 the temperature is within the desired range, the flow continues to step S214, where controller 74 evaluates whether the cooling duration is complete. For example, the cooling duration may be pre-programmed into controller 74, or may be provided by the user via user interface 78, prior to or at the time of initiating the cooling process. If the cooling duration is complete, controller 74 deflates the inflatable at step S216. Otherwise, the flow returns to step S202.

It will be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. Similarly, the content of a claim depending from one or more particular claims may generally depend from the other, unspecified claims, or be combined with the content thereof, absent any specific, manifest incompatibility therebetween.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A system for cooling a head of a user during medical treatments so as to mitigate or prevent drug-induced hair-loss, the system comprising a hat including:
an exterior layer;
an interior layer;
an inflatable chamber disposed between the exterior layer and the interior layer, the inflatable chamber being functionally associated with an inflation interface adapted for connection to an inflation pump;
a cooling assembly, reversibly attachable to an interior surface of the interior layer, the cooling assembly comprising a housing defining a plurality of pockets and a plurality of cooling pads, each comprising a phase-changing-material (PCM) adapted to obtain a predetermined temperature lower than −20° C. and to retain the predetermined temperature for a predetermined duration;
at least one sensor adapted to sense an environmental condition within the hat;
a first communication interface; and
a first controller, adapted to receive input from the at least one sensor, and to provide a notification to another device, via the first communication interface, when the sensed environmental condition is outside of a specific range,
wherein when the hat is donned by the user, the inflatable chamber is inflated sufficiently to ensure that the cooling assembly engages the scalp of the user.

2. The system of claim 1, wherein each cooling pad is adapted to obtain a predetermined temperature lower than −28° C.

3. The system of claim 1, wherein each cooling pad is adapted to retain the predetermined temperature of a duration of at least three hours.

4. The system of claim 1, wherein the at least one sensor comprises at least one temperature sensor adapted to provide to the first controller input regarding a temperature within the hat.

5. The system of claim 1, wherein the at least one sensor comprises at least one pressure sensor adapted to provide to the first controller input regarding a pressure applied by the hat to the scalp of the user.

6. The system of claim 1, wherein the hat further comprises a sizing assembly adapted to adjust at least one dimension of the hat so as to maintain the hat fixed on the user's head.

7. The system of claim 1, wherein the cooling assembly is devoid of fluid communication with other elements of the hat or outside the hat.

8. The system of claim 1, wherein the cooling assembly is devoid of electronic components.

9. The system of claim 1, further comprising a pumping unit, including:
a pump adapted to be in fluid communication with the inflation interface of the hat for inflation of the inflatable chamber;
a second communication interface adapted to be in communication with the first communication interface;
a user interface, adapted to receive input from a user and to provide output to the user; and
a second controller, functionally associated with the pump, the second communication interface, and the user interface, for controlling operation thereof.

10. The system of claim 9, wherein, upon the second controller receiving a signal from the first controller indicating that a temperature within the hat is above a predetermined temperature threshold, the second controller is adapted to provide to the user a notification, via the user interface, to replace the cooling pads in the cooling assembly.

11. The system of claim 9, wherein, upon the second controller receiving a signal from the first controller indicating that a pressure temperature within the hat is below a predetermined pressure threshold, the second controller is adapted to automatically activate the pump to inflate the inflatable chamber of the hat.

12. A device for control of a hat for cooling a head of a user, the hat having: (i) an inflatable chamber; (ii) a cooling assembly; (iii) a pressure sensor; (iv) a temperature sensor; and (v) a communication interface, the device comprising:
a pump adapted to be in fluid communication with the inflatable chamber of the hat;
a user interface adapted to provide output to the user; and
a controller, configured to:
receive temperature input from the temperature sensor; and
upon identification of the temperature input being indicative of a temperature in the hat being above a predetermined temperature threshold, provide a notification to the user, via the user interface, to replace cooling units of the cooling assembly within the hat.

13. The device of claim 12, wherein the controller is further configured to:
receive pressure input from the pressure sensor in the hat; and
upon identification of the pressure sensor being indicative of the pressure in the hat being below a predetermined pressure threshold, automatically activate the pump to re-inflate the inflatable chamber of the hat, thereby increasing the pressure therein.

14. A method of cooling the scalp of a user during a medical treatment so as to reduce or prevent drug-induced hair-loss, using the system of claim 1, the method comprising:
(a) at a first predetermined duration prior to initiation of the medical treatment, with the cooling assembly having cooling pads therein and being connected to the interior layer, donning the hat on the head of the user;
(b) inflating the inflatable chamber;
(c) retaining the hat on the head until receipt of a notification indicating that a temperature of the cooling assembly is above a temperature threshold;
(d) following (c), replacing the cooling pads within the cooling assembly and re-donning the hat on the head of the user;
(e) repeating steps (c) to (d) until the passage of a second predetermined duration following completion of the medical treatment.

15. The method of claim 14, wherein the first predetermined duration is at least one hour.

16. The method of claim 14, wherein the second predetermined duration is at least four hours.

17. The method of claim 14, wherein the temperature threshold is −20° C.

18. The method of claim 14, wherein the temperature threshold is −25° C.

19. The method of claim 14, wherein the temperature threshold is −28° C.
